(19) United States
(12) Patent Application Publication
(10) Pub. No.: US 2024/0075156 A1
(43) Pub. Date: Mar. 7, 2024

(54) TARGETING OF NEUTROPHIL EXTRACELLULAR TRAPS AND DIAGNOSTIC AND THERAPEUTIC USES THEREOF

(71) Applicants: Vladimir Torchilin, Charlestown, MA (US); Leonid Z. Iakoubov, Gresham, OR (US); Nina Filipczak, Boston, MA (US); Livia Palmerston Mendes, Tubingen (DE)

(72) Inventors: Vladimir Torchilin, Charlestown, MA (US); Leonid Z. Iakoubov, Gresham, OR (US); Nina Filipczak, Boston, MA (US); Livia Palmerston Mendes, Tubingen (DE)

(21) Appl. No.: 18/256,037

(22) PCT Filed: Dec. 6, 2021
(86) PCT No.: PCT/US2021/062050

§ 371 Date: Jun. 5, 2023

Related U.S. Application Data
(60) Provisional application No. 63/121,829, filed on Dec. 4, 2020.

(51) Int. Cl. A61K 47/68; A61K 49/00; A61P 37/02

(52) U.S. Cl. CPC A61K 47/6843; A61K 49/0002; A61P 37/02

(57) ABSTRACT

The present disclosure provides compositions for targeting neutrophil extracellular traps for diagnostic and therapeutic purposes (e.g., for treating inflammation in conditions or diseases associated with accumulation of neutrophil extracellular traps in a subject in need thereof).

### CROSS-REFERENCE TO RELATED APPLICATIONS

This application is a National Phase application under 35 U.S.C. 371 of PCT/US2021/062050, filed Dec. 6, 2021, which claims the benefit of and priority to U.S. Ser. No. 63/121,829, filed Dec. 4, 2020, all of which are incorporated herein by reference in their entirety.

### BACKGROUND OF THE INVENTION

Neutrophil extracellular traps (NETs), networks of extracellular fibers primarily consisting of decondensed chromatin, neutrophil elastase, and granular contents, were first described as a defense mechanism to trap and kill bacteria and other pathogens. Since nuclear chromatin bears various peptides and granule-derived proteins from parent neutrophils, sites of concentrated NETs are toxic to invading pathogens. NETs are, however, also responsible for collateral damage to the host in the setting of chronic NET or impaired NET removal. For instance, NETs are involved in acute thrombotic complications in cardiovascular and autoimmune disease, abnormal response to infection in systemic lupus erythematosus (SLE), and tumor metastasis. Moreover, excess NET formation generally jeopardizes normal endothelial function, and may contribute to organ damage and mortality. The problem of NET removal under conditions when their pathological role becomes dominant is clearly of utmost importance. Accordingly, there is a need for diagnostic and therapeutic agents targeting NETs.

### SUMMARY OF THE INVENTION

In one aspect, the disclosure provides a composition for targeting neutrophil extracellular traps (NETs), NET chromatin, or products of its degradation. The composition includes a targeting agent for NETs and an active agent or combination of agents. In some embodiments, the composition further includes a detectable label. Exemplary targeting agents include an antibody, antigen-binding fragment thereof, or aptamer. In one embodiment, the active agent is a nuclease, e.g., DNAse I, DNAse IL2, or DNAse IL3. In some embodiments, the targeting agent and active agent are formulated in a carrier, e.g., a liposome, a micelle, or a nanoparticle. In some embodiments, the active agent is conjugated to the targeting agent.

In a related aspect, the invention provides a method of treating a NET-related disease or condition in a subject by administering to the subject a composition as described herein. In embodiments, the condition or disease is an autoimmune disease, a cardiovascular disease, ischemia-reperfusion injury. In one embodiment, the condition or disease is an infection, e.g., bacterial, fungal, parasitic, or viral, e.g., caused by SARS-CoV-2 or a variant thereof. In one embodiment, the condition or disease is inflammation, e.g., sterile inflammation. In one embodiment, the condition or disease is a neoplasm. In one embodiment, the condition or disease includes thrombosis.

### BRIEF DESCRIPTION OF THE DRAWINGS

FIGS. 1A and B are a set of graphs showing immunoreactivity of monoclonal antibody (mAB) 2C5. Indirect ELISA was used to evaluate the binding of 2C5 antibody to a monolayer of different antigens. (A) HL-60 produced NETs and nucleohistones, and (B) neutrophils produced NETs and nucleohistones. The affinity for both substrates is similar, indicating 2C5 has specific activity against NETS, as well as for nucleosomes. Detection was done using a horseradish peroxidase (HRP)-conjugated secondary antibody. Data represents the mean±SD, n=3.

FIGS. 2A and B are a set of photomicrographs and graphs showing mAB 2C5 binding to NETs in vitro. (A) Neutrophils were isolated from whole blood and stimulated with either phorbol myristate acetate (PMA) or calcium ionophore (Cl), followed by staining with 2C5 antibodies and counterstaining using Hoechst. Activated neutrophils produced NETs, confirmed by the presence of web-like structures and filamentous structures containing decondensed DNA (arrows). These structures were also labeled with 2C5 to show the specificity of this antibody against NETs, presenting an overlap with the DNA staining (B) Pearson's correlation coefficient was analyzed using Image-J and indicates good correlation of pixel intensity distribution between channels in PMA and Cl treated groups. The image brightness and contrast of the 2C5 channel were adjusted using the B&C tool of Image-J Software to improve visualization, especially in the overlay images. Equal adjustments were applied to images that were obtained at the same day and using the same microscope parameters. Scale bar=50 μm.

FIGS. 3A and B are a set of photomicrographs from microfluidic studies illustrating 2C5-NET interactions. Representative widefield images of NETs formed in microfluidic channels, stained with SYTOX Orange, a DNA dye impermeable to live cells. The separate channels (included in the same visual field) were simultaneously infused at 2 dynes/cm² with Alexa 488-labeled 2C5 and non-specific lgG (A) or Alexa 488-labeled liposomes modified with either 2C5 or non-specific lgG on their surface (B). Arrows represent flow direction.

FIG. 4 is a set of photomicrographs showing that morphological changes in NETs do not prevent 2C5 binding. The addition of platelet factor 4 (PF4), a factor released by activated platelets in heparin-induced thrombocytopenia, induces chromatin compaction in NETs as noted by reduction in NET area. PF4-NET complex formation also influences NET behavior by inducing DNase I resistance. However, 2C5 binding is not prevented, indicating that the conformational epitope required for 2C5 binding is preserved and available despite morphological changes. Arrows represent flow direction.

FIGS. 5A and 5B are a set of graphs and photomicrographs showing HL-60 differentiation into neutrophil-like cells. (A) Nitroblue tetrazolium (NBT) assay. Production of formazan crystals indicates that HI-60 cells are transformed into neutrophil-like cells after exposure to dimethylsulfoxide (DMSO) or all-trans retinoic acid (ATRA) for different time points. Differentiated cells reduce NBT into formazan crystals in the presence of PMA (n=3). (B) Images of HL-60 cells exposed to DMSO or ATRA. Phase holographic images show differences in size and morphology of cells differentiated in the presence of ATRA or DMSO for 3 days. Cell volume was analyzed with Hstudio analysis software (Phase Holographic Imaging) at 4 h and 72 h after incubation with the differentiation agents. The graphs for each group and time-point are presented below the images, showing changes in cell size after differentiation. Scale bar=100 μm.

FIGS. 6A-6C are a set of photomicrographs showing visualization of NETs by immunofluorescence. DMSO-dHL-60 (A), neutrophils isolated from whole blood stimulated with either 100 nM PMA or 4 μM Cl (B) and neutrophils stimulated with either 100 ng/mL 11-8 or 4 μg/mL LPS (C) and stained with FITC-labeled myeloperoxidase (MPO) antibody and counterstained with Hoechst 33342 for DNA and nuclei identification. In the case of non-stimulated (control) cells, MPO staining is restricted to the cytoplasm surrounding the nuclei, whereas for stimulated cells, MPO can be also found in the NET filaments and spread to the web-like structures. The image brightness and contrast of the MPO channel were adjusted using the B&C tool of Image-J software to improve visualization, especially in the overlay images. Equal adjustments were applied to images that were obtained on the same day and using the same microscope parameters. Scale bar=50 μm.

FIGS. 7A-7D are a set of graphs and photomicrographs showing NET quantification and visualization using SYTOX green. (A) dHL-60 cells and were treated with either 4 pM or 25 pM of the calcium ionophore A23187 and incubated for up to 3 h at 37° C. for NET stimulation. Unstimulated cells were used as control. The fluorometric quantification was based on the staining of extracellular DNA with 1 PM of SYTOX green (a cell impermeable dye), whose signal was acquired every 10 min over the 3 hours (mean±SD). (B) Images of extracellular DNA and cells with compromised cell membrane stained using 5 PM SYTOX green after 3 h of stimulation of dHL-60 with 4 PM of Cl (live unfixed cells). Scale bar=50 μm. (C) neutrophils cells treated with either 100 vg/mL LPS or 100 ng/mL of the IL-8 and incubated for up to 3 h at 37° C. for NET stimulation. Unstimulated cells were used as control. The fluorometric quantification was based on the staining of extracellular DNA with 1 PM of SYTOX green (a cell impermeable dye), whose signal was acquired every 10 min over 15 the 3 hours (mean±SD). (D) Images of extracellular DNA and cells with compromised cell membrane stained using 5 PM SYTOX green after 3 h of stimulation of isolated neutrophils with 100 μg/mL of LPS (live unfixed cells). Scale bar=50 μm.

FIGS. 8A-8I are images of activity of conjugates. The activity of a PE-PEG-pNP:DNAse conjugate and DNAse I-2C5 conjugate in degrading the nucleohistone. A solution of the nucleohistone was incubated with DNase I (free or conjugated) for various time-points at 37° C. Samples were analyzed in 1% SYBR Safe Gel (15 μL per well at 60 mV for 16 min). (A) Free Nucleohistone; (B) Free DNAse I; (C) DNase I+ Nucleohistone; (D) DNAse I conjugate with PE-PEG-pNP (1.5:1 molar ratio); (E) DNAse I conjugate with PE-PEG-pNP (3:1 molar ratio); (F) DNAse I conjugate with PE-PEG-pNP (6:1 molar ratio); (G) DNAse I-2C5 conjugate (1.5:1, molar ratio); (H) DNAse I-2C5 conjugate (3:1 molar ratio); (I) DNAse I-2C5 conjugate (6:1 molar ratio).

### DETAILED DESCRIPTION OF THE INVENTION

This invention is based, in part, on the surprising discovery that compositions (e.g., conjugate, liposome, micelle, or nanoparticle) including a neutrophil extracellular trap (NET)-targeting agent (e.g., monoclonal antibody (mAB 2C5) and an active agent (e.g., a nuclease such as DNAse I) allow for specific visualization and degradation of NETs. The compositions described herein may reduce the abnormal accumulation of NETs in subjects suffering from NET-related pathologies (e.g., autoimmune conditions, cancers, cardiovascular diseases, infections, and sterile inflammation with or without thrombotic events).

### I. Definitions

As used herein, the term “administer” refers to the placement of a composition into a subject by a method or route which results in at least partial localization of the composition at a desired site such that desired effect is produced. A compound or composition described herein can be administered by any appropriate route known in the art, including, but not limited to, intravenous, intramuscular, subcutaneous, transdermal, airway (e.g., aerosol), pulmonary, nasal, rectal, and topical (e.g., buccal and sublingual) administration. Exemplary modes of administration include, but are not limited to, injection, infusion, instillation, inhalation, or ingestion. “Injection” includes, without limitation, intravenous, intramuscular, intraarterial, intraventricular, intracapsular, intracardiac, intradermal, intraperitoneal, transtracheal, subcutaneous, intraarticular, and intrastemal injection and infusion.

As used herein, the term “antibody” refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, recombinant IgG (rlgG) fragments, and scFv fragments. Moreover, unless otherwise indicated, the term “monoclonal antibody” (mAB) is meant to include both intact molecules, as well as antibody fragments (such as, for example, Fab and F(ab′) 2 fragments) that are capable of specifically binding to a target protein.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment of the V_{H} and C_{H}1 domains; (iv) a Fv fragment of the V_{L} and V_{H} domains of a single arm of an antibody, (v) a dAb including V_{H} and V_{L} domains; (vi) a dAb fragment (Ward et al., Nature 341:544-546, 1989) of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “aptamer” refers to a targeting agent that binds to various molecular targets including small molecules, proteins, nucleic acids, cells, tissues, and organisms. Aptamers may be nucleic acid aptamers (e.g., DNA aptamers or RNA aptamers) or peptide aptamers. Nucleic acid aptamers refer generally to nucleic acid species that are engineered through in vitro selection or an equivalent method (e.g., systematic evolution of ligands by exponential enrichment (SELEX)) to bind specific molecular targets. Peptide aptamers typically include a variable peptide loop attached at both ends to a protein scaffold that increases the binding affinity of the peptide aptamer to levels comparable to those of an antibody with the same intended molecular target.

As used herein, the term “conjugate” refers to a compound formed by the chemical bonding of a reactive functional group of one molecule with an appropriately reactive functional group of another molecule. Conjugates may additionally be produced, e.g., as two polypeptide domains covalently bound to one another as part of a single polypeptide chain that is synthesized by the translation of a single RNA transcript encoding both polypeptides in frame with one another.

As used herein, the term “humanized” antibody refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and 6,180,370; EP239400; PCT publication WO 91/09967; U.S. Pat. No. 5,225,539; EP592106; and EP519596.

The word “label” when used herein refers to a detectable compound or composition which is conjugated directly or indirectly to the antibody so as to generate a “labeled” antibody. The label may be detectable by itself (e.g., radioisotope labels, magnetic resonance labels, or fluorescent labels).

Whereas the terms “one or more” or “at least one,” such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the term “pharmaceutical carrier” refers to a pharmaceutically acceptable material, composition, or vehicle (e.g., lipid nanoparticle, micelle, or vesicle) involved in carrying or transporting the treatment agents from one organ or portion of the body to another organ or portion of the body.

As used herein, the term a “subject” is a mammal Mammals include, but are not limited to, domesticated animals (e.g., cows, sheep, cats, dogs, and horses), primates (e.g., humans and non-human primates such as monkeys), rabbits, deer, and rodents (e.g., mice and rats). In some embodiments, the subject is a human.

As used herein, the terms “treat” and “treatment” refer to therapeutic treatment, in which the object is to prevent the progression of or slow down (lessen) the severity of a NET-related condition or disease, such as systemic lupus erythematosus, by reducing the concentration of NETs, NET chromatin, and/or NET degradation products in blood products or the bloodstream. Beneficial or desired clinical results include, but are not limited to, the alleviation of autoimmune symptoms associated with abnormal NET accumulation.

Neutrophil extracellular traps (NETs) include processed chromatin bound to granular and select cytoplasmic proteins that are released by neutrophils (e.g., neutrophil elastase (NE) and myeloperoxidase (MPO)) to control microbial infections. NETs have a backbone of smooth filaments composed of stacked nucleosomes loaded with globular domains of granular proteins. Fully hydrated NETs have a web-like appearance and cover a space 10-1 5-fold larger than the volume of the cells from which they originate.

At least two mechanisms of NET release exist. One mechanism requires the lytic suicide of cells, while the other, vital NETosis, does not require the rupture of the cytoplasmic membrane. A variety of stimuli induce NETosis, ranging from pathogens, to activated platelets (in a TLR-4-dependent mechanism), anti-neutrophil cytoplasmic antibodies, and artificial compounds such as phorbol myristate acetate (PMA) and bacterial lipopolysaccharides.

Although NETs were first described as a defense mechanism to trap and kill bacteria and other pathogens, they are implicated as key players in numerous disease states, including cancer and cardiovascular disease. For example, NETs released by tumor associated neutrophils protect tumor cells from the immune cytotoxic effects of cytotoxic T lymphocytes and natural killer cells by preventing their direct contact 12. Furthermore, NET inhibition re-sensitizes the tumor cells to the immune cytotoxicity. Metastatic cancer cells also can induce neutrophils to form metastasis-supporting NETs. NET-like structures are also observed around metastatic 4T1 cancer cells that had infiltrated the lungs of mice.

NET chromatin. Nucleosomes are individual units of chromatin that include approximately 147 nucleotides of DNA wrapped around a histone octamer core. When NETs are formed, their chromatin is decondensed and pushed into extracellular space such that multiple series of stacked nucleosomes are exposed to the bloodstream. During this process, other proteins such as NE and MPO bind to the decondensed chromatin.

Products of NET degradation. Degradation products of NETs include nucleosomes, myeloperoxidase (MPO), neutrophil elastase (NE), citrullinated Histone 3 (citH3), cell-free DNA (cfDNA), and the association of these components, such as MPO-cfDNA.

### II. Compositions

The present disclosure provides compositions to detect and/or reduce NET accumulation in a subject (e.g., a human) The compositions include a targeting agent (e.g., an antibody) and an active agent (e.g., a nuclease) to bind and degrade NETs, NET chromatin, and/or products of NET degradation. In some embodiments, the targeting agent is directly conjugated to the active agent via a covalent bond or spacer. In some embodiments, the compositions are encapsulated in a pharmaceutical carrier (e.g., a liposome, micelle, or nanoparticle). In some embodiments, the compositions further include a label, such as a fluorescent label or radiolabel.

Targeting Agents

Targeting agents of the disclosure direct the compositions to NETs, NET chromatin, and/or products of NET degradation. These agents may be antibodies, antigen-binding fragments thereof, aptamers, or a combination thereof.

Antibodies, including antigen-binding fragments and single chain versions, against fragments or components of a NET can be raised by immunization of animals with conjugates of the fragments with carrier proteins. Monoclonal antibodies are prepared from cells secreting the desired antibody. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281. These antibodies can be screened for binding to NETs or their ability to disrupt NETs as described herein. These monoclonal antibodies will usually bind with at least an equilibrium dissociation constant (K_{D}) of about 1 mM, e.g., at least about 300 μM, 100 μM, 30 μM, 10 μM, 3 μM, 1 μM, 0.3 μM, 0.1 μM, or better.

Antibodies and antigen-binding fragments described herein include fully human, humanized, primatized, and chimeric antibodies that recognize and bind to one or more (e.g., two, three, four, or five) components of NETs. In some embodiments, the antibody or antigen-binding fragments described herein recognize NETs. In some embodiments, the antibody or antigen-binding fragments described herein recognize stacked nucleosomes. In some embodiments, the antibody or antigen-binding fragments described herein recognize histone proteins. In some embodiments, the antibody or antigen-binding fragments described herein recognize DNA. In some embodiments, the antibody or antigen-binding fragments described herein recognize NE. In some embodiments, the antibody or antigen-binding fragments described herein recognize MPO. In some embodiments, the antibody or antigen-binding fragments described herein recognize products of NET degradation. Examples of antibodies that target NETs include 2C5 and IG3.

Aptamers are also included as targeting agents. Examples of aptamers include nucleic acid aptamers (e.g., DNA aptamers, RNA aptamers) and peptide aptamers. In some embodiments, the aptamer can bind to one or more (e.g., two, three, four, or five) components of the NETs. In some embodiments, the aptamer can bind to NET chromatin. In some embodiments, the aptamer can bind to products of NET degradation.

Active Agents

Active agents of the disclosure may degrade NETs, NET-chromatin, and/or products of NET degradation.

Exemplary active agents include, but are not limited to, nucleases, histone-degrading enzymes, and heparin. Nucleases are enzymes that cleave phosphodiester bonds between nucleotides of nucleic acids. Nucleases (e.g., DNAse I, DNAse I-like 2, and DNAse I-like 3) can be used to degrade NETs. In some embodiments of the disclosure, a nuclease is included in the pharmaceutical carrier. In some embodiments, the nuclease is a DNAse. In some embodiments, the nuclease is DNAse I. In some embodiments, the nuclease is DNAse II. In some embodiments, the DNAse is DNAse III. In some embodiments, the nuclease is an RNAse. In some embodiments, the nuclease is an exonuclease. In some embodiments, the nuclease is an endonuclease. Histone-degrading enzymes are proteins capable of histone proteolysis. In some embodiments of the disclosure, a histone-degrading enzyme is the active agent. In some embodiments, the histone-degrading enzyme is Mast Cell Proteinase 1, Plasmin, Cathepsin D, or Activated Protein C (APC). In some embodiments, the active agent is heparin.

Labels

The compositions described herein may be labeled by covalently or non-covalently joining a substance which provides a detectable signal. A variety of labels and conjugation techniques are known and extensively reported in both scientific and patent literature. Suitable labels for the compositions described herein include fluorescent labels, magnetic resonance labels, and radiolabels. Specific examples include Cy, rhodamine, fluorescein, FITC, carboxyfluorescein, 1-121, In-111, Tc-99m, Gd, and Mn.

Pharmaceutical Carriers

In some embodiments, the compositions described herein are in the form of pharmaceutical carriers, including, but not limited to, liposomes (e.g., PEGylated), micelles (e.g., PEG-PE), nanoparticles (e.g., lipid nanoparticles), mesoporous silica, and dendrimers. Methods for producing carriers are known in the art. In some embodiments, the carrier includes dipalmitoylphosphatidylcholine (DPPC), 1,2distearoyl-sn-glycero-3-phosphoethanol amine-N-lazido(PEG)-2000 (azide-terminated PEG2000-DSPE), and/or 1-palmitoyl-2-(dipyrromethene boron difluoride)undecanoyl-sn-glycero-3-phosphocholine (Top fluor PC). The NET targeting agent and active agent may be conjugated or unconjugated when in a carrier.

Conjugates

In some embodiments, the active agent (e.g., DNAse I) is directly conjugated to the targeting agent (e.g., mAB 2C5) by a linker. The linker may be any suitable structure including a conjugation moiety (e.g., alkyl linkers or poly(alkylene oxide), such as a poly(ethylene) glycol (PEG) polymer, such as p-nitrophenylcarbonyl-polyethyleneglycol-phosphatidylethanolamine (pNP-PEG-PE)) or an amino acid sequence (e.g., a 1-200 amino acid sequence) occurring between the active agent and the targeting agent to provide space and/or flexibility between the two agents. A conjugation moiety includes at least one functional group (e.g., diamines, disulfides, SPDP, dimethyl suberimidate, N-hydroxysuccinimide-ester, aryl azides) that is capable of undergoing a conjugation reaction (e.g., a cycloaddition reaction (e.g., dipolar cycloaddition)), amidation reaction, nucleophilic aromatic substitution). The conjugation moiety may be protected until the active agent is conjugated to the targeting agent.

Additional Components

The compositions of the invention may further include or be combined with additional therapeutic agents. Such additional agents may act to reduce NET formation or to provide a different therapeutic modality for treatment of the disease or condition at hand. In some embodiments, a thrombolytic agent, such as urokinase, may additionally be added to the composition or combined in a formulation with a composition.

Compositions may also further include or be combined with a PAD4 inhibitor, e.g., Cl-amidine. Other agents that may be included are chloroquine, neutrophil elastase inhibitors, tissue plasminogen activator (tPA) and anti-PD1 or anti-PDLL

### III. Pharmaceutical Formulations and Methods of Administration

The compositions are preferably formulated into pharmaceutical formulations for administration to human subjects in a biologically compatible form suitable for administration in vivo. In accordance with the methods of the invention, the described compositions may be administered to a patient in a variety of forms depending on the selected route of administration, as will be understood by those skilled in the art. The compositions of the invention may be administered, for example, by oral, parenteral, buccal, sublingual, nasal, rectal, patch, pump, or transdermal administration and the pharmaceutical formulations prepared accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A composition of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a composition of the invention may be incorporated with an excipient and used in the form of ingestible tablets, buccal tablets, troches, capsules, elixirs, suspensions, syrups, wafers, and the like.

A composition of the invention may also be administered parenterally. Solutions of a composition of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in in Remington: The Science and Practice of Pharmacy, 23^{rd} Ed., Adej are, Ed., Academic Press (2020) and in The United States Pharmacopeia and National Formulary (USP 43 NF38), published in 2019.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that may be easily administered via syringe.

Formulations for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge of refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer

Formulations suitable for buccal or sublingual administration include tablets, lozenges, and pastilles, where the active ingredient is formulated with a carrier, such as sugar, acacia, tragacanth, gelatin, and glycerin. Formulations for rectal administration are conveniently in the form of suppositories containing a conventional suppository base, such as cocoa butter.

The compositions of the invention may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

A composition may be formulated with a pharmaceutically acceptable diluent. The diluent may include, but is not limited to, a sterile saline solution, sterile water, and/or phosphate-buffered saline (PBS). The diluent is typically of pharmaceutical grade.

In some embodiments, pharmaceutical formulations include one or more (e.g., two, three, four, or five) excipients. In some embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In some embodiments, compositions described herein may be admixed with pharmaceutically acceptable diluents for the preparation of pharmaceutical formulations. Compositions and methods for such pharmaceutical formulations depend on a number of criteria, including, but not limited to, route of administration, extent of disease, or dose to be administered.

### IV. Methods of Use

The present disclosure provides a method to aid in treating, detecting, diagnosing, preventing, or ameliorating a disease or medical condition associated with abnormal accumulation of NETs in a subject, or where NETs have a harmful effect on the subject or prevent healing or a normal process in the subject, such as an immune response. These indications are termed “NET-related.” Accordingly, the invention provides a method for treating inflammation in subjects with NET-related conditions or diseases (e.g., autoimmune diseases (e.g., systemic lupus erythematosus, rheumatoid arthritis, or psoriasis), cardiovascular diseases (e.g., atherosclerosis, vascular occlusion, or thrombosis, e.g., as a complication of atherosclerosis), ischemia and reperfusion injury (e.g., from myocardial infarction or organ transplantation), neoplasm, kidney disease, pancreatitis, wounds (e.g., burns or ulcers), infections, or inflammation (sterile or infectious with or without thrombotic events).

The method includes administering to a subject an effective amount of the composition containing an active agent, a targeting agent, and optionally a label (e.g., fluorescent label or radiolabel) to treat or diagnose a subject with a NET-related condition or disease. The composition can target NETs, NET chromatin, and/or products of NET degradation and deliver DNAse and/or other active agents to degrade the NETs, NET chromatin, and/or products of NET degradation. This approach can be therapeutically or diagnostically useful in many pathological conditions, including cancer, cardiovascular diseases, and viral infections including COVID-19.

In some embodiments, the NET-related condition or disease is an autoimmune disease, e.g., systemic lupus erythematosus, rheumatoid arthritis, or psoriasis. In some embodiments, the NET-related condition or disease is a neoplasm. In some embodiments, the NET-related condition or disease is cardiovascular disease, e.g., atherosclerosis, vascular occlusion, or thrombosis. In some embodiments, the NET-related condition or disease is an infection, e.g., bacterial, viral, parasitic, or fungal. In some embodiments, the infection is viral. In some embodiments, the viral infection is caused by SARS-CoV-2 or a variant thereof. In some embodiments, the NET-related condition is inflammation, e.g., sterile or infectious. In some embodiments, the sterile inflammation includes thrombotic events. In some embodiments, the sterile inflammation does not include thrombotic events. In some embodiments, the NET-related condition or disease is ischemia-reperfusion injury, e.g., from myocardial infarction or organ transplantation. In some embodiments, the NET-related condition or disease is kidney disease. In some embodiments, the NET-related condition or disease is pancreatitis. In some embodiments, the NET-related condition or disease is a wound, e.g., burns or ulcers.

The dosage ranges for the administration of the compositions described herein depend on the form of the compound, its potency, and the extent to which marker or symptoms of a condition described herein are desired to be reduced, for example the percentage reduction desired for inflammation or thrombus size. The dosage should not be so large as to cause adverse side effects such as pulmonary edema or congestive heart failure. Generally, the dosage will vary with the age, condition, and sex of the patient and can be determined by one of skill in the art. The dosage can also be adjusted by the individual physician in the event of any complication. The administration may be performed once or repeated at regular intervals, for example, biweekly for two months. In some embodiments, the administration may be on a less frequent basis after an initial treatment regimen to maintain the reduced serum levels of NETs, NET chromatin, and products of NET degradation. Administration of the compositions described herein can reduce levels of a marker or symptom of a condition or disease described herein, e.g., inflammation, by at least 10% (e.g., at least 15%, at least 20%, at least 25%, at least 30%, at least 35%, at least 40%, at least 45%, at least 50%, or more).

The presence or disruption of NETS can be monitored in vivo or in vitro. In one embodiment, the presence or disruption of NETS is monitored by assessing the level of NET release in stored blood in the presence and absence of a test compound (e.g., by ELISA and/or determination of DNA concentration) as described herein. In one embodiment, the ability of a test compound to disrupt NETs is monitored in vivo as described herein and/or determining the ability to prevent thrombosis or protect against stroke as described herein. Compositions of the invention, e.g., those including a label, may also be employed in the detection of NETs, e.g., for the purpose of diagnosing a condition or monitoring the efficacy of treatment.

### V. EXAMPLES

The following example is put forth to provide those of ordinary skill in the art with a description of how the compositions and methods described herein may be used and evaluated and is intended to be purely exemplary of the invention and is not intended to limit the scope of what the inventors regard as their invention.

### Example 1: HL-60 Differentiation and Characterization

Materials and Methods

### Materials & Methods

### Materials

Anti-myeloperoxidase (MPO) primary antibody (sc-51741 FITC) and a goat anti-mouse lgG phycoerythrin-labeled secondary antibody (sc-3738) were purchased from Santa Cruz Biotechnology. Monoclonal antibody (mAB) 2C5 was produced by Harlan Bioproducts. Hoechst 33342 (H3570) was from Molecular Probes. HL-60 cells and Iscove's Modified Dulbecco's Medium (IMDM) were purchased from ATCC (ATCC@ 30-2003 m). Lymphocyte separation media (MT25072CI) and RPMI 1640 (MTI 0040CM) were acquired from Corning and RBC lysis buffer 1×(00433357) was from Invitrogen. Dimethyl sulfoxide (DMSO) (AA439985Y) and dextran (MW 250,000 g/mol) (AAJ6020009) were from Alfa Aesar. Nitro blue tetrazolium (NBT) (BPI 08-1) was from Fisher Scientific. Phorbol myristate acetate (PMA) (NC9750026) was from LC Laboratories. All-trans retinoic acid (ATRA) (AC207341000) and A23187 (AC328080010) were purchased from Acros Organics. SYTOX green nucleic acid stain (S7020) was from Thermo Fisher Scientific. Recombinant human interleukin-8 (BD 554609) was from BD Bioscience. LPS from E. coli (TLRLEBLPS) was from Invivogen. Poly-L-lysine hydrobromide (MVV 30,000-70,000) (P2636-25MG) was purchased from Sigma. Calf thymus nucleohistone (LS00301 1) was acquired from Worthington Biochemical Corporation. 1,2-dipalmitoyl-snglycero-3-phosphocholine (DPPC) (850355), 1-palmitoyl-2-(dipyrrometheneboron ifluoride)undecanoyl-sn-glycero-3-phosphocholine (Top fluor PC) (810281) and 1,2distearoyl-sn-glycero-3-phospho ethanolamine-N-lazido(polyethylene glycol)-2000 (azide-terminated PEG2000-DSPE) (880228) were purchased from Avanti. All other reagents were of analytical grade.

### Cell Culture and Differentiation

To develop an in vitro model of neutrophil extracellular traps (NETs), a human promyelocytic leukemia cell line, HL-60, was differentiated into neutrophil-like cells by agents including all-trans retinoic acid (ATRA) and dimethyl sulfoxide (DMSO)³¹. HL-60 cells were cultured using IMDM medium supplemented with 10% fetal bovine serum (FBS) and, 100 U/mL penicillin and 100 μg/mL streptomycin were added at 37° C., 5% CO 2 and humidified air. For differentiation, these cells were exposed to ATRA and DMSO at 1 PM and 125%, respectively, for 3 to 5 days. To confirm the differentiation, cells were analyzed at various time-points by the nitro blue tetrazolium (NBT) reduction method^{32,33} time-lapse phase holographic imaging³⁴ and by brightfield microscopy (Keyence, BZ-X700, Itasca, IL, USA).

Human neutrophils were also used to produce NETs. For that, neutrophils were first isolated from whole blood 35 using a density gradient separation technique. Briefly, blood was collected from healthy volunteers in heparinized tubes and kept on ice until use. Blood was diluted with phosphate buffer saline (PBS) pH 7.4 (without calcium and magnesium) and layered on top of Lymphocyte Separation Media (I-SM—density 1.077 g/mL). Tubes were centrifuged at 800×g for 30 min at 21 C. The top two layers (monocytes and lymphocytes) were removed. Dextran (MVV 250,000 g/mol) was added and mixed in tubes at a final concentration of 3%. The suspension was allowed to rest for 30 min at 37° C. to sediment the red blood cells (RBC) and the supernatant containing the granulocytes were transferred to a new tube. Tubes were centrifuged at 450×g at 4° C. for 5 min The supernatant was discarded and a 1× RBC lysing solution was added to the tube to re-suspend the pellets and lyse any remaining RBC. The tubes were kept in the dark for 10 min, centrifuged at 450×g at 4° C. for 5 min The supernatant containing lysed RBC was discarded. The pellet containing isolated neutrophils was washed by re-suspension in 5 mL PBS, centrifuged again, resuspended with RPMI medium supplemented with 3% PBS, and used immediately.

### NBT Reduction Assay

A quantitative NBT assay^{36,37} with modifications was used to determine the differentiation of HL-60 into granulocyte-like cells. Control HL-60 and cells were differentiated for different periods of time (3 to 5 days). DMSO-dHL-60 and ATRA-dHL-60 were seeded in 96-well plates (200,000 cells/well) and incubated with NBT at 1 mg/mL in the presence or absence of 10 μg/mL of PMA. After 30 min incubation, the formation of formazan crystals was observed under the microscope. The plate was centrifuged to recover the crystals, supernatant was removed, and the crystals were solubilized with DMSO. The absorbance of the solution was measured at 620 nm using a microplate reader (Synergy H T, 25 Biotek, VT, USA) and the increase in absorbance (indicating the formation of crystals) was compared to that of control HL-60.

### Phase Holographic Imaging

Phase Holographic Imaging (PHI) is a laser-based methodology that captures timelapse images of unlabeled cells and allows for observation and quantification of differences in cell morphology based on holographic reconstructions of the cells' refractive index^{34,38}. For holographic microscopy, cells were seeded on glass-bottom, 24-well plates and incubated in the presence or absence of ATRA and DMSO. The Holomonitor M4 (Phase Holographic Imaging, Lund, Sweden) was used to collect images every 10 minutes over 5 days from each well (3 regions/well) using a 20× objective lens. The data collected was analyzed using Hstudio analysis software (Phase Holographic Imaging) to characterize the morphological features of cells in the differentiation process.

### NET Quantification

For NET quantification, the presence of extracellular DNA was analyzed^{4,31}. Differentiated cells were seeded in 96-well plate (100,000 cells/well) and stimulated with the calcium ionophore A23187 (CD at 4 PM and 25 PM for up to 3 hours at 37° C. to induce production of NETs. The presence of extracellular DNA release was quantified by addition of 5 1 1-1M SYTOX Green (DNA stain impermeant to live cells) followed by fluorometric analysis (excitation/emission: 504/523 nm) every 10 minutes. Unstimulated cells were used as controls to compare the increase in the fluorescent signal in the presence of the Cl at different time points.

A similar experiment was conducted with the use of the isolated neutrophils. After 10 seeding neutrophils were stimulated with LPS from E. coli (100 pg/mL) and IL-8 (100 ng/mL) for up to 3 hours at 37° C. to induce production of NETs.

### NET Visualization

NETs have been described as threads of chromatin containing nuclear content and cytoplasmic proteins, including MPO and NE⁴. Hence, NET visualization was accomplished by immunofluorescence based on staining of the cells with antibodies against MPO and DNA. Neutrophils and HL-60 cells were seeded on glass-bottom 24-well plates and stimulated with PMA (100 nm) and calcium ionophore (CI) (4 μM) for 4 hours and with LPS (100 μg/mL) and IL-8 (100 ng/mL) for 3 hours at 37° C. to induce NET production. Cells were fixed with 4% paraformaldehyde (PFA), followed by blocking to prevent non-specific binding. The plates were incubated with FITC-labeled primary anti-MPO antibody at a 1:100 dilution, washed, and counterstained with Hoechst 33342 to visualize the presence of NETs released from stimulated cells. The staining patterns of PMA-, CI-, IL-8-, and LPS-stimulated cells were compared to those of unstimulated cells. In addition, live unfixed cells were labeled with SYTOX Green to observe the presence of extracellular DNA release and cells with compromised cell membranes, signaled by the presence of the internalized DNA stain. The labeled plates were observed by epigluorescence microscopy (Keyence, BZ-X700). Image-J software 1.50a (NIH, Bethesda, MD) with FIJI package 39 was used to adjust the brightness and contrast of the images in the MPO channel using the B&C tool to improve visualization of the structures, especially in the overlay images. Equal adjustments were applied to images that were obtained on the same day using the same microscope parameters.

Specific Activity of Monoclonal Antibody (mAB) 2C5

An ELISA assay was used to verify the activity of mAB 2C5 using NETs isolated from Cl-stimulated HL-60 cells and from IL-8 stimulated neutrophils as a substrate. For NET isolation, 35 differentiated HL-60 (dHL60) in serum-free IMDM were seeded at 1 5 million cells/mL, followed by stimulation with 4 PM of Cl for 4 h. Neutrophils were incubated with 100 ng/mL of IL-8 for 3 h in RPMI medium supplemented with 3% serum.

The medium was gently removed, and cold PBS was added by thorough pipetting to remove any material adhered to the bottom of the flask. The suspension containing cells and NETs was collected and centrifuged at 450×g for 10 min at 4° C. to pellet the cells. NET-enriched cell-free supernatant was collected for use as a substrate in the ELISA assay. For isolation of NETs from stimulated neutrophils, additional centrifugation of NET-enriched cell-free supernatant was conducted at 16000×g for 10 min at 4° C. to pellet the NETS, which were used as a substrate for the ELISA assay.

Briefly, an ELISA plate was first coated with poly-L-lysine (PIX). Either nucleosomes (calf thymus nucleohistone) or NET-enriched medium diluted in TBST-Casein solution were added to attach to the PLI-layer and incubated for 1 h at room temperature (RT). The wells were washed 3 times with TBST and a serial dilution of 2C5 was added to the wells and incubated for 1 h at RT. The samples were removed, and the wells were washed 3 times with TBST. A secondary anti-mouse HRP antibody was added to the plate and incubated for 2 h at RT, followed by 3× washing of the wells. The chromogenic 3,3,5,5-tetramethylbenzidine (T MB) substrate was added and incubated up to 15 min, followed by microplate reading at either 450 nm or 652 nm, depending on the instructions of the T MB reagent used in each assay.

### Liposome Preparation

Liposomes functionalized with 2C5 or isotype control lgG were prepared as previously described 44. Lipid films were prepared by nitrogen drying and lyophilization of a mixture of chloroform solutions of DPPC:cholesterol:azide-terminated PEG2000-DSPE:Top Fluor PC (57.5:40:2:0.5 mol %). Dry films were hydrated with PBS and suspensions were formed via alternating bath sonication at 50° C. and vortexing at RT. Lipid suspensions were submitted to three freeze-thaw cycles via liquid N2 and 50° C. water bath.

Final suspensions were extruded through 200 nm polycarbonate filters. Liposome size and concentration following extrusion were evaluated via dynamic light scattering and NanoSight nanoparticle tracking analysis, respectively.

2C5 and isotype-matching control lgG were prepared for conjugation to azide liposomes by modification with dibenzocyclooctyne-PEG4-n-hydroxysuccinimidyl ester (DBCO). Briefly, antibody solutions were adjusted to pH 8.3 with 1M NaHCO₃buffer and reacted with DBCO at a molar ratio of 1 antibody:5 DBCO for 1 h at RT. Unreacted DBCO was removed via filtration against a 10 kDa cutoff centrifugal filter. DBCO optical absorbance (peak at 309 nm) was compared to antibody optical absorbance (shoulder at 280 nm) in order to verify DBCO conjugation to antibodies.

DBCO-modified antibodies were conjugated to azide liposomes by overnight RT incubation at a ratio of 200 antibody molecules per liposome. Free antibodies were separated from liposomes via size exclusion chromatography. Liposomes were concentrated to original liposome volume with centrifugal filters and subsequently characterized with dynamic light scattering using a ZS90 Malvern Zetasizer Nano (Malvern, Westborough, MA). Liposomes had a size of 150 nm and Pd1 of 0.1.

### 2C5 Staining of NETs

The ability of 2C5 to bind NETs was evaluated in two different in vitro models using neutrophils isolated from whole blood as described previously. In the first model, 250,000 neutrophils were seeded directly on glass coverslips (previously coated with PIX), allowed to attach for 1 h, and stimulated for 4 h with either 100 nM of PMA or 4 PM of Cl in a CO2 incubator at 37° C. Medium was removed and the cells were fixed with 4% paraformaldehyde at RT for 10 min, followed by permeabilization with 0.25% Triton X-100 (v/v) in PBS. After washing the cells 3 times, 5% (w/v) bovine serum albumin was added and cells were allowed to incubate overnight at 4° C. After removing the blocking solution, 2C5 primary antibody was added at 5 μg/mL and the coverslips were incubated for 1 h at RT. The samples were washed 3 times with PBS and a phycoerythrin-labeled goat anti-mouse antibody was added (1:200), followed by 1 h incubation at RT. After washing the cells 3 times with PBS, Hoechst was added for counterstaining and the coverslips were mounted on glass slides with mounting medium. The slides were analyzed using the Keyence BZ-X700 epifluorescence microscope. Image-J with FIJI package was again used to adjust the brightness and contrast of the images in the 2C5 channel using the B&C tool to improve visualization of the structures, especially in the overlay images. Equal adjustments were applied to images that were obtained at the same day and using the same microscope parameters. In addition, the Coloc 2 plugin from FIJI was also used to analyze the images for the Pearson's correlation coefficient (PCC) for colocalization analysis of pixels distribution intensity between 2C5 and DNA staining.

A NET-Iined microfluidic study was also performed following the procedure described previously. 45 Briefly, a BioFlux 200 Controller (Fluxion) was used and the channels were visualized with an Axio Observer Zl inverted microscope (Zeiss) equipped with a motorized stage and an HXP-120 C metal halide illumination source. A 2 million cells/mL cell suspension was prepared using isolated neutrophils stimulated with tumor necrosis factor-alpha (TNFα) (Gibco, 10 ng/mL) and flowed through fibronectin-coated channels to which they adhere. The cells were incubated overnight at 37° C. in the presence of 100 nM of PMA to release NETs. The channels were then flowed at 2 dynes/cm² with 1 PM of SYTOX Orange to stain cell-free DNA. The 2C5 antibody was conjugated to Alexa Fluor 488 using EDC-NHS synthesis. Isotype lgG was prepared in the same conditions. Samples of fluorescently labeled liposomes modified with either 2C5 or isotype lgG (described in item 2.8) were used in the analysis. Both antibodies and liposomes were infused through the BioFlux channels at 2 dynes/cm² and both static images and videos were obtained. NET staining by 2C5 was analyzed using the videos in which channels with 2C5 and isotype lgG (free or conjugated to liposomes) were included in the same visual field. In addition, platelet factor 4 (PF4) was infused in the channels. PF4-NET complexes show a different morphology, with compact NETs. The ability of 2C5 to bind these complexes after morphological changes was also evaluated. Images obtained as Z-stacks were processed using Image-J with FIJI package and Z-projections using maximum pixel intensity were created to better reflect the overall staining of the NETs in the plate's channels. All procedures were approved by the IRB.

### Statistical Analysis

Results are expressed as mean±standard deviation of three experiments performed independently. Analysis of variance test (ANOVA) followed by Tukey's multiple comparisons test were used for comparison between groups unless otherwise indicated. Statistical difference was accepted when p<0.05. Statistical analysis was done using GraphPad Prism software (version 6.01 for Windows, GraphPad Software).

Results

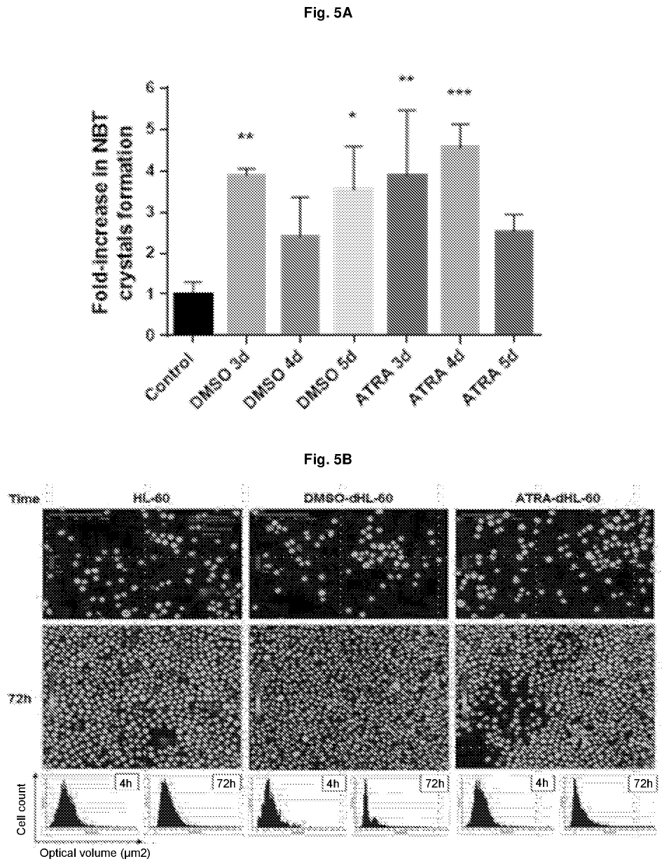

To facilitate the studies of NETs and their properties, NETs or NET-like structures were generated in vitro by exposing the human promyelocytic leukemia cell line to dimethyl sulfoxide (DMSO) and all-trans retinoic acid (ATRA), resulting in the differentiation of these cells into granulocyte-like cells (FIG. 5A). Results from the NBT reduction assay (FIG. 5A) show that after exposure to both ATRA and DMSO for as little as 3 days, dHL-60 cells were able to reduce nitro blue tetrazolium (NBT) into formazan crystals in the presence of PMA, a common characteristic of 30 granulocytes. The extent of crystal formation was similar for both groups, which produced approximately 4-fold more formazan crystals than control HL-60 cells after 3 days of differentiation.

Differences in the morphology of HL-60 cells exposed to DMSO or ATRA after 3 days were observed (FIG. 5B), although they were more pronounced in DMSO-differentiated cells. The volume of the cells decreased when incubated in the presence of both compounds. Additionally, DMSO differentiated cells (DMSO-dHL-60) shifted to a more angular shape.

NET Stimulation, Visualization, and Quantification

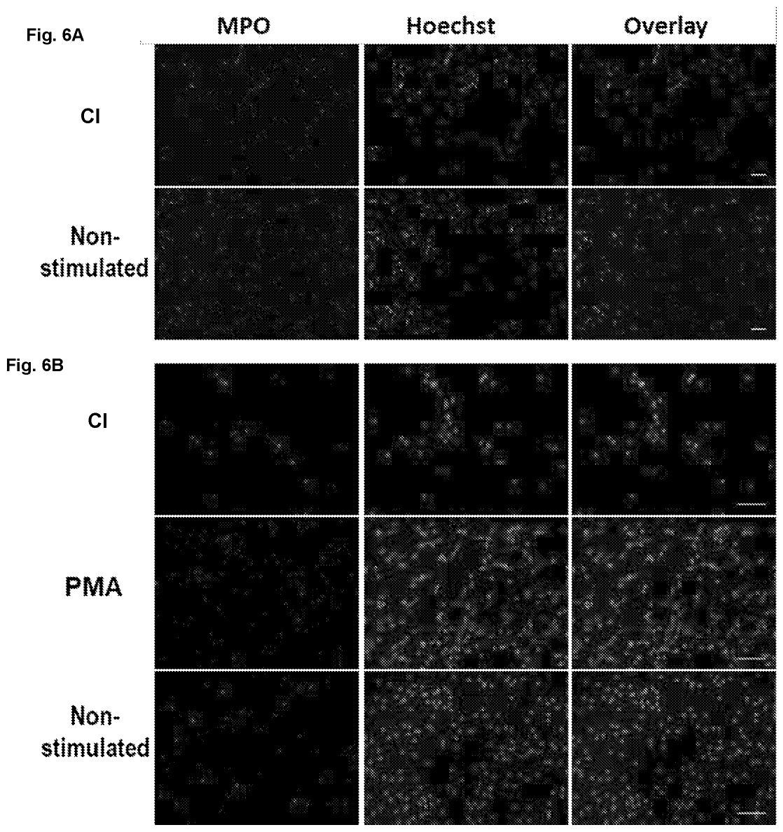

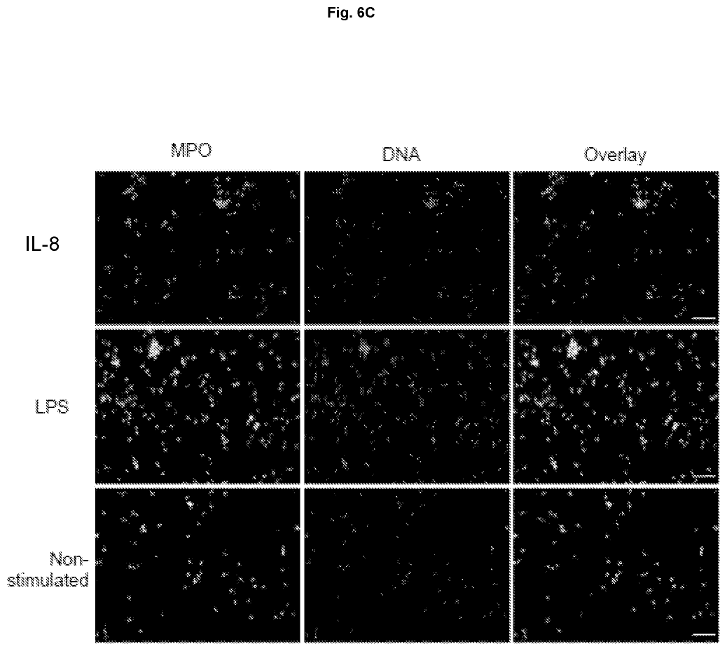

Neutrophils can generate NETs in vitro upon stimulation with certain compounds⁸. The web-like structures of chromatin-encompassing proteins expressed in granulocytes, such as myeloperoxidase (MPO) and neutrophil elastase (NE), can be visualized with fluorescent microscopy after staining the DNA and granular proteins^{4,46}. Differentiated HL-60 also extrude decondensed genomic DNAs into the extracellular space that are morphologically similar to NETs from neutrophils stimulated with LPS or II-8. Generation of NETs from both differentiated HL-60 cells and neutrophils, visualized by staining MPO can be seen in FIG. 6. DMSO-dHL-60 could generate more visible NETs when incubated with the calcium ionophore (CI) A1 8723 rather than with phorbol myristate acetate (PMA) or ATRA-dHL-60 exposed to Cl or PMA (data not shown). For this reason, all subsequent assays were done using DMSO-dHL60 differentiated cells.

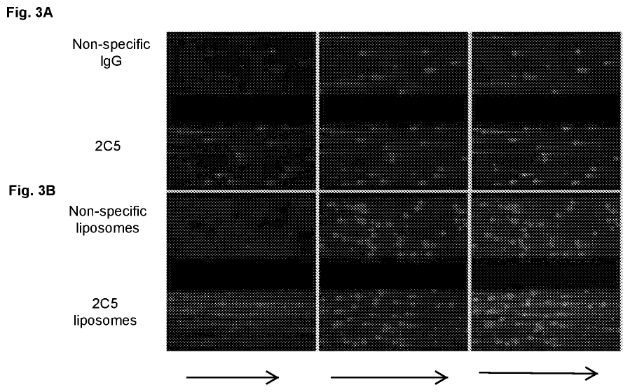

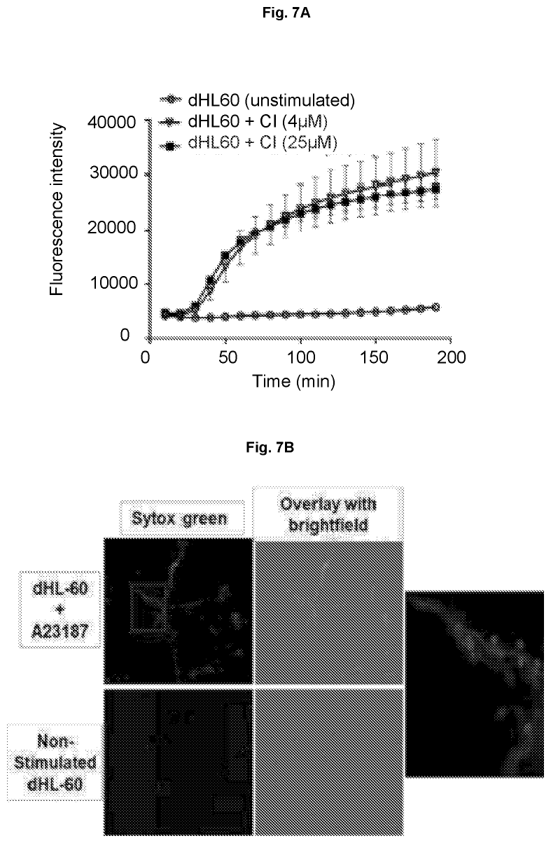

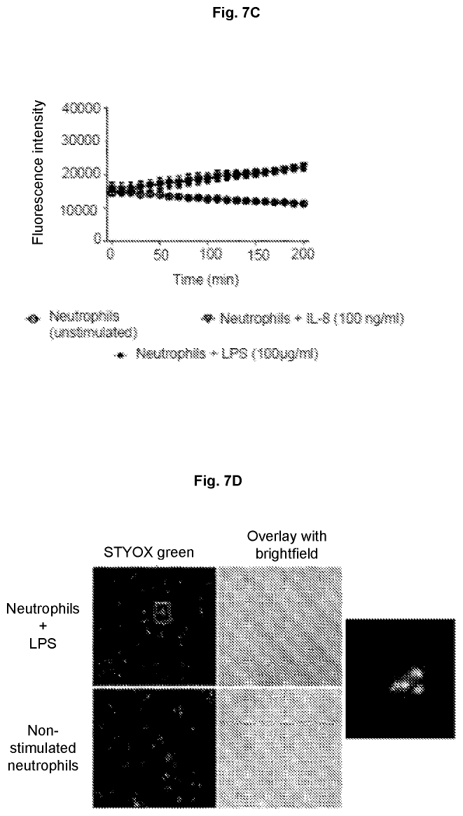

Quantification of NETs by fluorometric analysis was achieved by measuring the fluorescent signal of cells stimulated with Cl and stained with SYTOX green, a DNA dye impermeable to live cells (FIG. 7). The fluorescence observed thus represents DNA released from the cells or that of membrane-compromised cells. FIGS. 3A and 3C show the kinetic pattern of the increase in green fluorescence from the groups stimulated with Cl, 11-8, and LPS over 3 h. The fluorescence from the stimulated cells was clearly higher than that of non-stimulated cells, although there is some difference in the kinetic pattern for neutrophils and differentiated HL-60 cells. The use of a higher concentration of Cl did not promote more DNA release Imaging techniques were compared to the NET quantification to confirm the fluorescence signal is from generated NETs. As can be seen in FIG. 3B, following Cl-stimulation of dHL-60 cells, NETs are formed, but fluorescence originates not only from extruded NETs, but also from DNA still within the boundaries of the cell membrane. FIG. 3D illustrates NETs formation in neutrophils following LPS stimulation. Although cells and stimulants are different, the response of the cells resulting in NETs formation seems to be very similar.

Specific Activity of Monoclonal Antibody (mAB) 2C5

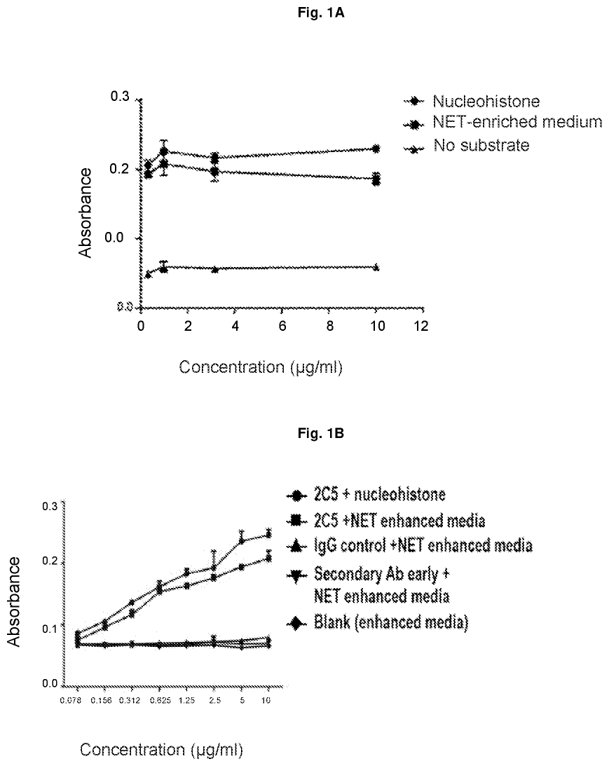

ELISA is widely used to assess the binding of antibodies to antigens immobilized on a solid surface. To verify the immunoreactivity of mAB 2C5 against NETs in a pattern similar to nucleosomes, a NET-enriched supernatant isolated from stimulated dHL-60 was used as a substrate to coat an ELISA plate using the same procedure as done for nucleohistone coating⁴¹⁻⁴³. FIG. 1 shows that mAB 2C5 bound effectively to the NET monolayer to a degree similar to that of nucleohistones, suggesting that 2C5 presents specific activity against NETs.

### 2C5 Binds to NETs in Different In Vitro Models

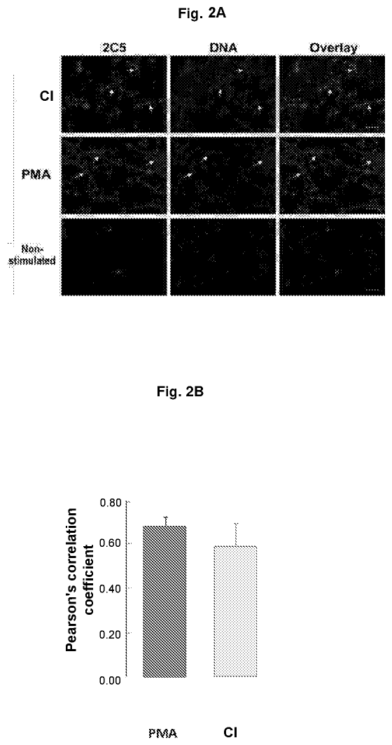

Given that mAB 2C5 showed specific activity to NETs, its capacity to stain these structures for microscopy purposes was assessed. Neutrophils isolated from whole blood were activated by incubation with Cl and PMA to generate NETs. As shown in FIG. 2, activated neutrophils released filaments and diffuse, cloudy structures (FIG. 2, arrows) that readily stained with the Hoechst DNA dye. This finding was most prominent in the PMA-stimulated group, although also noteworthy in Cl-stimulated neutrophils. When 2C5 was added, it successfully stained NETs, showing a co-localization of 2C5 and DNA staining in the overlap images. The co-localization was assessed visually and by obtaining the Pearson's correlation coefficient (PCC) and indicates there is good correlation of pixel intensity distribution between red and blue channels in PMA- and Cl-treated groups. The considerable PCC values in these groups indicate that 2C5 co-localizes with DNA in the NET structures formed upon neutrophil stimulation.

A microfluidic cell culture system was also used to evaluate 2C5 binding to NETs. Fibronectin-coated microfluidic channels were infused with neutrophils activated with TNFα. Following channel wall adhesion, the cells were stimulated with PMA to generate NETs. NETs were stained with SYTOX orange to identify the extracellular DNA, and the channels were infused with either Alexa 488-labeled 2C5 and or isotype lgG to evaluate the antibody specificity under conditions of flow. FIG. 3A shows that 2C5 antibody readily binds and stains NETs, while its isotype counterpart does not bind to the extracellular chromatin. The same pattern was observed for liposomes modified with 2C5 (FIG. 3B), with an apparently even higher interaction than with antibody alone. The web-like structure of NETs could allow for the non-specific capture of these macromolecules, but the 5 specificity of 2C5 binding is clearly shown by its stronger staining signal.

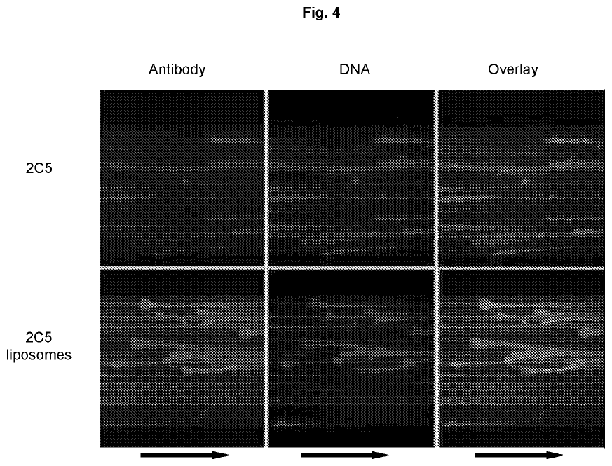

To further assess if 2C5 can bind NETs under variable conditions, platelet factor 4 (PF4) was perfused in the microfluidic channels. PF4 is released by activated platelets in high concentrations at sites of thrombosis and in heparin-induced thrombocytopenia (HIT). Due to its positive charge, it binds to the extracellular DNA released by NETs, forming compact PF4-NET complexes (FIG. 4). Despite these morphological changes, 2C5 binding was still achieved for both the antibody alone and the decorated liposomes, indicating that the conformational epitope required for 2C5 binding is preserved and remains available.

Cell differentiation was assessed by distinct methods including microscopy to observe cell morphology, a fluorescent plate assay to confirm the release of NETS, and the functional NBT reduction assay. The latter is based on the conversion of oxygen into superoxide anion by NADPH oxidase, which can then reduce NBT leading to the formation of blue crystals^{36,37}. Other studies have similarly shown changes in the respiratory burst of differentiated cells. In these studies, however, ATRA-dHL-60 reduced NBT more efficiently than DMSO and other compounds^{31,47}. Longer incubation times did not significantly increase the generation of the crystals, indicating that 3 days are sufficient to obtain cells with neutrophil-like characteristics. After differentiation, the cells were stimulated to check their capacity to generate NETs, followed by staining for visualization. It is known, however, that only about 10% of dHL-60 can release NET-like structures after stimulation with a variety of compounds. In addition, the released structures are shorter and more discreet than the long and diffuse NETs released by neutrophils^{48,49}, a characteristic also observed in this study (FIG. 6). Manda-Handzlik and co-workers showed that different agents differentiate HL-60 by distinct mechanisms, affecting their ability to generate NETs in response to various stimuli. They also found that DMSO-dHL-60 released NET-like structures only after stimulation with Cl, but not with PMA³¹. In addition, NET generation from neutrophils can also be induced by variety of stimuli. Natural triggers for NETosis include bacteria or endogenous stimuli, such as immune complexes. As an experimental counterpart for calcium ionophore, LPS from E. coli and interleukin-8 were used to demonstrate different pathway of NETosis.⁹

NETs were quantified by the fluorescence analysis. While this method provides insight into DNA release, it is not NET-specific 5° as Cl can increase dHL-60 membrane permeability, 51 facilitating intracellular DNA staining with SYTOX. Hence, imaging techniques were used as a confirmatory technique, showing that although SYTOX also stained intracellular DNA in Cl-stimulated cells, the formation of NET-like structures was clear (FIGS. 7A-7B). Lelliott, et al. recently developed an imaging flow cytometry automated analysis method to characterize and quantify NETs in vitro based on DNA staining⁵². In their study, they characterized different cell types and NET structures after exposure to NET stimulants. Besides the extracellular DNA (exDNA) and DNA fragments characteristic of NETs, they noticed the presence of SYTOX-stained cells with decondensed DNA and an absence of exDNA. They hypothesized that these cells represent another type of cell death rather than NETosis.

The specific activity of 2C5 towards NETs was evaluated using ELISA, which showed that the antibody binds to NET enriched substrate, obtained from interleukin-8 stimulated neutrophils or Cl-stimulated dHL-60 cells. mAB 2C5 is a nucleosome-specific monoclonal antibody and was shown to recognize only intact NS among many nuclear and other antigens. These mABs possessed NS-restricted specificity in ELISA when tested against artificially reconstituted NSs²⁶⁻²⁸ mABs with selective specificity for NS, i.e. to chromatin, the main component of NETs, have been described earlier by Yager et al⁵³. Using ELISA to test a panel of different nuclear antigens, their specificity towards NSs was clearly demonstrated. In ELISA and Western blot assays with the isotype-matched UPC10 antibody as a negative control, they did not react with such nuclear antigens as ssDNA, dsDNA, histones (individual and mixtures), HI peptide 144-159, HI peptide 204-218, ribonucleoprotein, La/SS-B, Ro/SS-A, Sm, Jo-1, and scl-70. It also did not react with other tested antigens including myosin, b-galactosidase, phosphorylase b, glutamic dehydrogenase, lactate dehydrogenase, carbonic anhydrase, trypsin inhibitor, lysozyme, aprotinin, insulin, heparin, heparin sulfate, and dextran sulfate. These mABs were shown to possess intact NS-restricted specificity in ELISA when tested against artificially reconstituted NSS.^{54,55} Individual components of NSs, DNA and histones, were not recognized by them as well as the mixture of the DNA and histones that did not contain the reconstituted NSs (a stepwise salt dialysis procedure was not applied to DNA/histone mixture)⁵³. DNAse treatment eliminates mAB 2C5 binding with NS²⁷. Control isotype-matched anti-myeloma UPC10 antibody does not react with NSs. These mABs appear to recognize a conformational epitope of intact NS formed only by the fragments of histones 2 and 3. In ELISAs, mAB 2C5 demonstrated good binding with the mixture of histones 2 and 3 assembled on heparin (instead of DNA) (unpublished observation). The web-like structures, comprised mostly of the chromatin released from the cells, provide the binding sites for 2C5 in NETs. Therefore, 2C5 could be used as a tool to identify and target NETs using different 5 approaches.

The use of 2C5 as immunostaining for NETs was confirmed in vitro in a static and in a perfused microfluidic cell model. NET imaging is based largely on co-staining extracellular DNA and cytoplasmic proteins bound to the chromatin, including MPO, NE, and histone 3. Since it is known that 2C5 does not recognize individual components of NSs, including DNA and histones, its use as another staining approach to identify these structures may prove useful in other NET research fields. The microfluidic system was used in addition to the regular cell culture since the shear produced by the flow in this setup provides a different environment than that of NETs immobilized on a coverslip and can be set up to simulate flow rate as in vivo. In the microfluidic experiment, PF4 was added to the cells to analyze whether the formation of PF4-NET complexes, which causes NET compaction and morphological changes, impairs 2C5 binding to NETs. In addition, PF4 compaction of NETs also induces DNase I resistance⁴⁵, which could also take advantage of the use of targeted therapy. The formation of the complexes is a finding that also occurs with multiple other cations⁵⁶ and likely influences NET behavior in vivo. The fact that 2C5 can still bind to NETs after these morphology changes suggests that it would be useful as a targeting moiety in drug delivery approaches for different types of NETs and for different diseases, including those associated with intense platelet activation such as HIT, disseminated intravascular coagulation, and thrombotic storm.

In conclusion, promyelocytic leukemia cells could be differentiated into neutrophil-like cells, which were then used to generate NETs for in vitro assessment. The ability of 2C5 to bind to various NETs was confirmed by ELISA and immunostaining. The NS-restricted specificity of 2C5 allows recognition of NETs by these antibodies due to the presence of NS (chromatin fragments) in these structures, even after exposure to PF4, which promotes NET compaction and DNase resistance. Thus, 2C5 can be used to target NETs for diagnostic purposes (visualization) when modified with appropriate labels. Furthermore, the 2C5 antibody can be used to control a variety of pathologies associated with NETs accumulation, such as cardiovascular, inflammatory, autoimmune, and malignant diseases, by delivering NET-targeted pharmaceutical agents, including drug-loaded nanocarriers bearing agents for NET destruction. The use of NET-targeted pharmaceutical nanocarriers could be of special importance when more than one therapeutic agent is delivered. For example, NET-driven thromboses can be treated with a combination of nucleases that cleave DNA and site-directed thrombolytic agents, optimizing thrombus digestion. Such systems open unique new opportunities for the treatment of multiple pathologies.

### Example 2: mAB 2C5-Conjugated DNase 1 Preserves its Ability to Degrade Nucleosomes

### Materials and Methods

Carbamate linker was used for the attachment of amino acids (DNAse I and 2C5 antibody) via their N-termini Firstly, 32.2 mmol of DOPE was dissolved in chloroform to obtain a 50 mg/ml solution. The solution was supplemented with approximately 2-fold molar excess over PEG-(pNP)₂ of TEA. Then 10-fold molar excess over DOPE of different length PEG-(pNP)₂ dissolved in chloroform was added to the mixture, and the sample was incubated overnight at room temperature with stirring. Afterwards, organic solvents were removed using a rotary evaporator, and the pNP-PEG-DOPE micelles were formed in 0.01 M HCl, 0.15 M NaCl. The micelles were separated from the unbound PEG and released pNP on a CL-4B column using 0.01 M HCl, 0.15 M NaCl as an eluent. Pooled fractions containing pNP-PEG-DOPE were freeze-dried and stored.

pNP-PEG3Ak-PE and PEG2k-PE (molar ratio 1:40) solutions were used to form a film. Similarly, pNP-PEG2k-PE and PEG2k-PE (molar ratio 1:40) were mixed, evaporated, and freeze-dried. In next step DNase solution (0.5 mg/ml, solubilized in 0.1 sodium bicarbonate buffer) was added to pNP-PEG3Ak-PE film and stirred for 4 h at 4° C. Similarly, 2C5 solution (solubilized in PBS) was added to pNP-PEG7k-PE, the pH of the solution was adjusted to 8.0 with 0.1N of NaOH, and reaction was incubated overnight at 4° C. with gentle stirring. Afterwards, the solution was dialyzed (MWCO 300,000 Da) against PBS pH 7.4 at 4° C. for at least 4 h.

Finally, DNase-PEG3.4k-PE micelles and 2C5-PEG7k-PE micelles were mixed in a 40:1 ratio of DNase:2C5 and equilibrated overnight at 4° C. Results of Characterization of DNAse Conjugates

Immunoblot

The successful DNAse conjugation to PEG chain was determined by Micro BCA assay and followed by western blot analysis. Conjugate and free DNAse I were resolved by SOS-PAGE, transferred to a PVDF membrane, and probed with polyclonal horseradish peroxidase anti-DNase I antibodies. The blots were imaged in chemiluminescent solution on the ChemiDoc™ XRS+ System (Bio-Rad, Hercules, CA, US).

Gel Electrophoresis

A solution of the nucleohistone (nucleosomes), 2 ml with concentration of 1 mg/ml, was incubated with 1 μg of DNase I (free or conjugated) for various time-points (60, 120, and 240 min) at 37° C. in phosphate-buffered saline (pH 7.4). Samples were analyzed by gel-electrophoresis in 1% SYBR Safe Gel (15 μL per well at 60 mV for 16 min). DNase was modified with pNP-PEG at different ratios −1.5:1; 3:1, and 6:1 molar ratios, and conjugated with mAB 2C5 at different ratios −1.5:1, 3:1; 5 and 6:1 molar ratios.

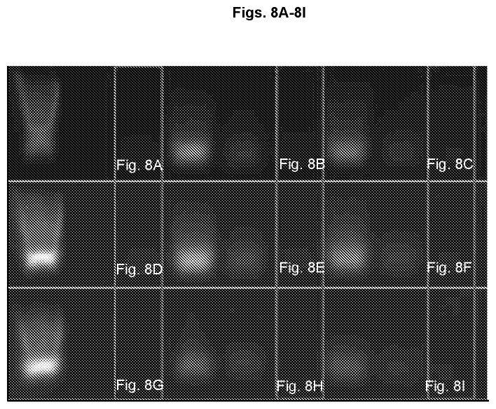

The results presented in FIG. 8A-81 demonstrate that mAB 2C5-conjugated DNase 1 preserves its ability to degrade nucleosomes, and similar preparations can be used in therapy involving the disintegration of NETs.

### REFERENCES

1. Sollberger G, Tilley D O, Zychlinsky A. Neutrophil Extracellular Traps: The Biology of Chromatin Externalization. Developmental cell 2018; 44:542-53.
2. Brinkmann V, Zychlinsky A. Neutrophil extracellular traps: is immunity the second function of chromatin? The Journal of cell biology 2012; 198:773-83.
3. Nauseef W M, Kubes P. Pondering neutrophil extracellular traps with healthy skepticism. Cellular microbiology 2016; 18:1349-57.
4. Brinkmann V, Reichard U, Goosmann C, Fauler B, Uhlemann Y, Weiss D S, et al. 25 Neutrophil Extracellular Traps Kill Bacteria. Science 2004; 303:1532.
5. Douda D N, Khan M A, Grasemann H, Palaniyar N. SK3 channel and mitochondrial ROS mediate NADPH oxidase-independent NETosis induced by calcium influx. Proc Natl Acad Sci USA 2015; 1 122817-22.
6. Yipp B G, Kubes P. NETosis: how vital is it? Blood 2013; 1222784-94.
7. Erpenbeck L, Schon M P. Neutrophil extracellular traps: protagonists of cancer progression? Oncogene 2017; 36:2483-90.
8. Hoppenbrouwers T, Autar A S A, Sultan A R, Abraham T E, van Cappellen W A, Houtsmuller A B, et al. In vitro induction of NETosis: Comprehensive live imaging comparison and systematic review. Plos one 2017; 12:e0176472.
9. Papayannopoulos V. Neutrophil extracellular traps in immunity and disease. Nature Reviews Immunology 2017; 18:134.
10. Jimenez-Alcazar M, Rangaswamy C, Panda R, Bitterling J, Simsek Y J, Long A T, et al. Host DNases prevent vascular occlusion by neutrophil extracellular traps. Science 2017; 358: 1202-6.
11. Park J, Wysocki R W, Amoozgar Z, Maiorino L, Fein M F R, Jorns J, et al. Cancer cells induce metastasis-supporting neutrophil extracellular DNA traps. Science translational medicine 2016; 8:361 ra138.
12. Teijeira A, Garasa S, Gato M, Alfaro C, Migueliz 1, Cirella A, et al. CXCRI and CXCR2 Chemokine Receptor Agonists Produced by Tumors Induce Neutrophil Extracellular Traps 45 that Interfere with Immune Cytotoxicity. Immunity 2020; 52.856-71.e8.
13. Franck G, Mawson T L, Folco E J, Molinaro R, Ruvkun V, Engelbertsen D, et al. Roles of PAD4 and NETosis in Experimental Atherosclerosis and Arterial Injury: Implications for Superficial Erosion. Circ Res 2018; 12333-42.
14. Qi H, Yang S, Zhang L. Neutrophil Extracellular Traps and Endothelial Dysfunction in 5 Atherosclerosis and Thrombosis. Frontiers in immunology 2017; 8:928.
15. Wang Y, Luo L, Braun 00, Westman J, Madhi R, Herwald H, et al. Neutrophil extracellular trap-microparticle complexes enhance thrombin generation via the intrinsic pathway of coagulation in mice. Scientific reports 2018; 8:4020.
16. Pertiwi K R, van der Wal A C, Pabittei D R, Mackaaij C, van Leeuwen M B, Li X, et al. 10 Neutrophil Extracellular Traps Participate in All Different Types of Thrombotic and Haemorrhagic Complications of Coronary Atherosclerosis. Thrombosis and haemostasis 2018; 1 18:1078-87.
17. Hakkim A, Furnrohr B G, Amann K, Laube B, Abed (J A, Brinkmann V, et al. Impairment of neutrophil extracellular trap degradation is associated with lupus nephritis. Proc Natl Acad 15 Sci USA 2010; 107:9813-8.
18. Albrengues J, Shields M A, Ng D, Park C G, Ambrico A, Poindexter M E, et al. Neutrophil extracellular traps produced during inflammation awaken dormant cancer cells in mice. Science 2018; 361:eaa04227.
19. Demers M, Krause D S, Schatzberg D, Martinod K, Voorhees J R, Fuchs T A, et al. Cancers predispose neutrophils to release extracellular DNA traps that contribute to cancer-associated thrombosis. Proceedings of the National Academy of Sciences 2012; 109: 13076.
20. Demers M, Wong S L, Martinod K, Gallant M, Cabral J E, Wang Y, et al. Priming of neutrophils toward NETosis promotes tumor growth. Oncoimmunology 2016; 5:el 134073.
21. Wen F, Shen A, Choi A, Gerner E W, Shi J. Extracellular DNA in pancreatic cancer 25 promotes cell invasion and metastasis. Cancer research 2013; 73:4256-66.
22. Barnes B J, Adrover J M, Baxter-Stoltzfus A, Borczuk A, Cools-Lartigue J, Crawford J M, et al. Targeting potential drivers of COVID-19: Neutrophil extracellular traps. J Exp Med 2020; 217:e20200652.
23. Koshkaryev A, Sawant R, Deshpande M, Torchilin V. Immunoconjugates and long 30 circulating systems: origins, current state of the art and future directions. Advanced drug delivery reviews 2013; 6524-35.
24. Gupta B, Levchenko T S, Mongayt D A, Torchilin V P. Monoclonal antibody 2C5mediated binding of liposomes to brain tumor cells in vitro and in subcutaneous tumor model in vivo. Journal of drug targeting 2005; 13:337-43.
25. Carter T, Mulholland P, Chester K. Antibody-targeted nanoparticles for cancer treatment. Immunotherapy 2016; 8:941-58.
26. lakoubov L, Rokhlin O, Torchilin V Anti-nuclear autoantibodies of the aged reactive against the surface of tumor but not normal cells Immunol Lett 1995; 47:147-9.
27. lakoubov L Z, Torchilin V P. A novel class of antitumor antibodies: nucleosome40 restricted antinuclear autoantibodies (ANA) from healthy aged nonautoimmune mice. Oncology research 1997; 9:439-46.
28. Torchilin V P, Lukyanov A N, Gao Z, Papahadjopoulos-Sternberg B. Immunomicelles: targeted pharmaceutical carriers for poorly soluble drugs. Proc Natl Acad Sci USA 2003; 100.6039-44.
29. Elbayoumi T A, Torchilin V P. Tumor-specific anti-nucleosome antibody improves therapeutic efficacy of doxorubicin-loaded long-circulating liposomes against primary and metastatic tumor in mice. Mol Pharm 2009; 6:246-54.
30. Erdogan S, Roby A, Torchilin V P Enhanced tumor visualization by gammascintigraphy with 1 1 1 In-labeled polychelating-polymer-containing immunoliposomes. Mol Pharm 2006; 3525-30.
31. Manda-Handzlik A, Bystrzycka W, Wachowska M, Sieczkowska S, StelmaszczykEmmel A, Demkow U, et al. The influence of agents differentiating HL-60 cells toward granulocyte-like cells on their ability to release neutrophil extracellular traps Immunology & Cell Biology 2018; 96:413-25.
32. Blair O C, Carbone R, Sartorelli A C. Differentiation of 1-11--60 promyelocytic leukemia cells monitored by flow cytometric measurement of nitro blue tetrazolium (NBT) reduction. Cytometry 1985; 6:54-61.
33. Jian P, Li Z W, Fang T Y, Jian W, Zhuan Z, Mei L X, et al. Retinoic acid induces 1-11--60 5 cell differentiation via the upregulation of miR-663. Journal of hematology & oncology 201 1′ 4:20.
34. Luther E, Mendes L P, Pan J, Costa D F, Torchilin V P. Applications of label-free, quantitative phase holographic imaging cytometry to the development of multi-specific nanoscale pharmaceutical formulations. Cytometry Part A 2017; 91:412-23.
35. Najmeh S, Cools-Lartigue J, Giannias B, Spicer J, Ferri I-E. Simplified Human Neutrophil Extracellular Traps (NETs) Isolation and Handling. J Vis Exp 2015:52687.
36. Choi H S, Kim J W, Cha Y N, Kim C A quantitative nitroblue tetrazolium assay for determining intracellular superoxide anion production in phagocytic cells. Journal of immunoassay & immunochemistry 2006; 27:31-44.
37. Racanicchi L, Montanucci P, Basta G P, Pensato A, Conti V, Calafiore R. Effect of all trans retinoic acid on lysosomal alpha-D-mannosidase activity in 1-11--60 cell: correlation with 1-11--60 cells differentiation. Molecular and cellular biochemistry 2008; 308:17-24.
38. Zhang Y, Sriraman S K, Kenny H A, Luther E, Torchilin V, Lengyel E. Reversal of Chemoresistance in Ovarian Cancer by Co-Delivery of a P-Glycoprotein Inhibitor and 20 Paclitaxel in a Liposomal Platform. Molecular Cancer Therapeutics 2016; 15:2282.
38. Schindelin J, Arganda-Carreras 1, Frise E, Kaynig V, Longair M, Pietzsch T, et al. FIJI: an open-source platform for biological-image analysis. Nature Methods 2012; 9:676-82.
39. Zou Y, Chen X, Xiao J, Bo Zhou D, Xiao Lu X, Li W, et al. Neutrophil extracellular traps promote lipopolysaccharide-induced airway inflammation and mucus hypersecretion in mice. Oncotarget 2018; 9: 13276-86.
41. Torchilin V P, Levchenko T S, Lukyanov A N, Khaw B A, Klibanov A L, Rammohan R, et al. p-Nitrophenylcarbonyl-PEG-PE-liposomes: fast and simple attachment of specific ligands, including monoclonal antibodies, to distal ends of PEG chains via p-nitrophenylcarbonyl groups. Biochimica et Biophysica Acta (BBA)—Biomembranes 2001; 151 1:397-41 1.
42. Lukyanov A N, Elbayoumi T A, Chakilam A R, Torchilin V P. Tumor-targeted liposomes: doxorubicin-loaded long-circulating liposomes modified with anti-cancer antibody. Journal of Controlled Release 2004; 100:135-44.
43. Koren E, Apte A, Jani A, Torchilin V P. Multifunctional PEGylated 2C5immunoliposomes containing pH-sensitive bonds and TAT peptide for enhanced tumor cell 35 internalization and cytotoxicity. J Control Release 2012; 160:264-73.
44. Hood E D, Greineder C F, Shuvaeva T, Walsh L, Villa C H, Muzykantov V R. Vascular Targeting of Radiolabeled Liposomes with Bio-Orthogonally Conjugated Ligands: Single Chain Fragments Provide Higher Specificity than Antibodies. Bioconjugate Chemistry 2018; 29:3626-37.
45. Gollomp K, Kim M, Johnston 1, Hayes V, Welsh J, Arepally G M, et al. Neutrophil accumulation and NET release contribute to thrombosis in HIT. JCI Insight 2018; 3.
46. Brinkmann V, Laube B, Abu Abed U, Goosmann C, Zychlinsky A. Neutrophil Extracellular Traps: How to Generate and Visualize Them. JoVE 2010:el 724.
47. Sham R L, Phatak P D, Belanger K A, Packman C H. Functional properties of HI-60 cells 45 matured with all-trans-retinoic acid and DMSO: Differences in response to interleukin-8 and fMLP. Leukemia Research 1995; 19:1-6.
48. Yaseen R, Blodkamp S, Ltithje P, Reuner F, Winger L, Naim H Y, et al. Antimicrobial activity of HL-60 cells compared to primary blood-derived neutrophils against Staphylococcus aureus. J Negat Results Biomed 2017; 16:2
49. Wang Y, Li M, Stadler S, Correll S, Li P, Wang D, et al. Histone hypercitrullination mediates chromatin decondensation and neutrophil extracellular trap formation. The Journal of cell biology 2009; 184:205-13.
50. van der Linden M, Westerlaken G H A, van der Vlist M, van Montfrans J, Meyaard L. Differential Signalling and Kinetics of Neutrophil Extracellular Trap Release Revealed by 55 Quantitative Live Imaging. Scientific reports 2017; 7:6529.
51. Jiang Z, Yin X, Jiang Q. Natural forms of vitamin E and 13-carboxychromanol, a longchain vitamin E metabolite, inhibit leukotriene generation from stimulated neutrophils by blocking calcium influx and suppressing 5-lipoxygenase activity, respectively. Journal of immunology 201 1; 186M 173-9.
52. Lelliott P M, Momota M, Lee M S J, Kuroda E, lijima N, Ishii K J, et al. Rapid Quantification of NE Ts In Vitro and in Whole Blood Samples by Imaging Flow Cytometry. Cytometry Part A 2019; 95565-78.
53. Yager T D, McMurray C T, Van Holde K E Salt-induced release of DNA from nucleosome core particles. Biochemistry 1989; 28:2271-81.
54. Peterson C L. Salt gradient dialysis reconstitution of nucleosomes. CSH protocols 2008; 2008:pdb.prot51 13.
55. Lee K M, Narlikar G. Assembly of nucleosomal templates by salt dialysis. Current protocols in molecular biology 2001; Chapter 21:Unit 21.6.
56. Lande R, Botti E, Jandus C, Dojcinovic D, Fanelli G, Conrad C, et al. The antimicrobial peptide 1.1.37 is a T-cell autoantigen in psoriasis. Nat Commun 2014; 5:5621.

1-18. (canceled)

19. A composition for use in treatment of neutrophil extracellular traps (NETs), NET original chromatin, or products of its degradation,
the composition comprising a selective targeting agent for NETs selected from the group of agents selectively binding to NET consisting of antibodies, antigen-binding fragments thereof, and aptamer
bound to an active agent or combination of agents for treatment of a condition or disease associated with NETs, or a carrier comprising the active agent,
in an effective amount to reduce the concentration of NETs, NET chromatin, and/or NET degradation products.

20. The composition of claim 19, further comprising a detectable label.

21. The composition of claim 19, wherein the targeting agent is selected from the group consisting of chimeric antibodies, humanized antibodies, heteroconjugate antibodies and antigen-binding fragments of antibodies.

22. The composition of claim 19 further comprising a thrombolytic agent, a PAD4 inhibitor, chloroquine, neutrophil elastase inhibitors, tissue plasminogen activator (tPA) and anti-PD1 or anti-PDL1.

23. The composition of claim 19, wherein the active agent is a nuclease.

24. The composition of claim 19, wherein the nuclease is DNAse I, DNAse IL2, or DNAse IL3.

25. The composition of claim 19, wherein the targeting agent and active agent are formulated in a carrier.

26. The composition of claim 19, wherein the carrier is a liposome, a micelle, or a nanoparticle.

27. The composition of claim 19, wherein the active agent is conjugated to the targeting agent

28. A method of treating a NET-related condition or disease in a subject, the method comprising administering to the subject the composition of claim 1 in an amount effective to reduce the abnormal accumulation of NETs in subjects suffering from NET-related pathologies.

29. The method of claim 28, wherein the condition or disease is an autoimmune disease, a cardiovascular disease, or ischemia-reperfusion injury.

30. The method of claim 28, wherein the condition or disease is an infection.

31. The method of claim 30, wherein the infection is bacterial, fungal, parasitic, or viral.

32. The method of claim 31, wherein the viral infection is caused by SARS-CoV-2 or a variant thereof.

33. The method of claim 28, wherein the condition or disease is inflammation, sterile inflammation, a neoplasm, or comprises thrombosis.
